(19) United States
(12) Patent Application Publication
(10) Pub. No.: US 2024/0245930 A1
(43) Pub. Date: Jul. 25, 2024

(54) PARTICLE DOSE OPTIMIZATION FOR PARTICLE ARC THERAPY

(71) Applicant: Elekta, Inc., Atlanta, GA (US)

(72) Inventor: Stuart Julian Swerdloff, Dunedin (NZ)

(21) Appl. No.: 18/559,991

(22) PCT Filed: May 13, 2022
(86) PCT No.: PCT/US2022/072315

§ 371 Date: Nov. 9, 2023

Related U.S. Application Data
(60) Provisional application No. 63/201,802, filed on May 13, 2021.

(51) Int. Cl. A61N 5/10

(52) U.S. Cl. CPC A61N 5/1031; A61N 5/1043; A61N 5/1071; A61N 5/1082; A61N 2005/1087; A61N 2005/1095

(57) ABSTRACT

Systems and techniques may be used to generate a radiotherapy treatment plan to execute using a particle beam from a continuously rotating gantry towards a target. A technique may include identifying a target location within a tumor of a patient, providing a particle beam configured to deliver radiotherapy treatment to the tumor along a trajectory using at least two energies including a first energy and a second energy, the first energy greater than the second energy, and determining a first location along the trajectory past the target location and a second location before the target location along the trajectory. The technique may include determining a configuration for the particle beam to deliver the first energy to the first location and the second energy to the second location. In some examples, a radiotherapy treatment plan according to the configuration may be output.

### CLAIM OF PRIORITY

This application claims the benefit of priority to U.S. Provisional Application No. 63/201,802, filed May 13, 2021, titled “METHOD FOR OPTIMIZING PARTICLE DOSE FOR PARTICLE ARC THERAPY,” which is hereby incorporated herein by reference in its entirety.

### BACKGROUND

Radiation therapy or “radiotherapy” may be used to treat cancers or other ailments in mammalian (e.g., human and animal) tissue. One such radiotherapy technique is referred to as “gamma knife,” by which a patient is irradiated using a number of lower-intensity gamma rays that converge with higher intensity and high precision at a targeted region (e.g., a tumor). In another example, radiotherapy is provided using a linear accelerator (“linac”), whereby a targeted region is irradiated by high-energy particles (e.g., electrons, high-energy photons, and the like). In another example, radiotherapy is provided using a heavy charged particle accelerator (e.g. protons, carbon ions, and the like), The placement and dose of the radiation beam is accurately controlled to provide a prescribed dose of radiation to the targeted region. The radiation beam is also generally controlled to reduce or minimize damage to surrounding healthy tissue, such as may be referred to as “organ(s) at risk” (OARs). Radiation may be referred to as “prescribed” because generally a physician orders a predefined dose of radiation to be delivered to a targeted region such as a tumor.

Generally, ionizing radiation in the form of a collimated beam is directed from an external radiation source toward a patient. Modulation of a radiation beam may be provided by one or more attenuators or collimators (e.g., a multi-leaf collimator). The intensity and shape of the radiation beam may be adjusted by collimation avoid damaging healthy tissue (e.g., OARs) adjacent to the targeted tissue by conforming the projected beam to a profile of the targeted tissue.

The treatment planning procedure may include using a three-dimensional image of the patient to identify the target region (e.g., the tumor) and such as to identify critical organs near the tumor. Creation of a treatment plan may be a time consuming process where a planner tries to comply with various treatment objectives or constraints (e.g., dose volume histogram (DVH) objectives or other constraints), such as taking into account importance (e.g., weighting) of respective constraints in order to produce a treatment plan that is clinically acceptable. This task may be a time-consuming trial-and-error process that is complicated by the various organs at risk (OARs) because as the number of OARs increases (e.g., about thirteen for a head-and-neck treatment), so does the complexity of the process. OARs distant from a tumor may be more easily spared from radiation, but OARs close to or overlapping a target tumor may be more difficult to spare from radiation exposure during treatment.

Generally, for each patient, an initial treatment plan may be generated in an “offline” manner. The treatment plan may be developed well before radiation therapy is delivered, such as using one or more medical imaging techniques. Imaging information may include, for example, images from X-rays, Computed Tomography (CT), nuclear magnetic resonance (MR), positron emission tomography (PET), single-photon emission computed tomography (SPECT), or ultrasound. A health care provider, such as a physician, may use three-dimensional imaging information indicative of the patient anatomy to identify one or more target tumors along with the organs at risk near the tumor. The health care provider may delineate the target tumor that is to receive a prescribed radiation dose using a manual technique, and the health care provider may similarly delineate nearby tissue, such as organs, at risk of damage from the radiation treatment.

Alternatively or additionally, an automated tool (e.g., ABAS® provided by Elekta AB, Sweden) may be used to assist in identifying or delineating the target tumor and organs at risk. A radiation therapy treatment plan (“treatment plan”) may then be created using an optimization technique based on clinical and dosimetric objectives and constraints (e.g., the maximum, minimum, and mean doses of radiation to the tumor and critical organs).

The treatment planning procedure may include using a three-dimensional image of the patient to identify the target region (e.g., the tumor) and to identify critical organs near the tumor. Image acquisition may be performed just before initiation of delivery of a specified radiation therapy fraction. Such imaging may provide information helpful for identifying a position of a target region or for identifying motion of the target region. Such contemporaneous imaging may be referred to generically as “real-time,” but in general a latency or time delay exists between an acquisition of an image and a delivery of radiation therapy.

Creation of a treatment plan may be a time consuming process where a planner tries to comply with various treatment objectives or constraints (e.g., dose volume histogram (DVH) objectives), taking into account their individual importance (e.g., weighting) in order to produce a treatment plan that is clinically acceptable. This task may be a time-consuming trial-and-error process that is complicated by the various organs at risk (OARs) because as the number of OARs increases (e.g., up to thirteen for a head-and-neck treatment), so does the complexity of the process. OARs distant from a tumor may be easily spared from radiation, while OARs close to or overlapping a target tumor may be difficult to spare.

The treatment plan may then be later executed by positioning the patient and delivering the prescribed radiation therapy. The radiation therapy treatment plan may include dose “fractioning,” whereby a sequence of radiation therapy deliveries are provided over a predetermined period of time (e.g., 45 fractions or some other total count of fractions), such as with each therapy delivery including a specified fraction of a total prescribed dose. During treatment, the position of the patient or the position of the target region in relation to the treatment beam is important because such positioning in part determines whether the target region or healthy tissue is irradiated.

In one approach, radiation therapy may be provided by using particles, such as protons (e.g., instead of photons). This typically may be referred to as proton therapy. One significant known advantage of proton therapy is it provides superior dose distribution with minimal exit dose compared to other forms of radiation therapy, such as x-ray therapy. There is a significant reduction of dose to organs at risk (OAR) because of the minimal exit dose. Further advantages include lower dose per treatment, which lowers the risk of side effects and may improve quality of life during and after proton therapy treatment.

One method of providing proton therapy is to use a broad proton beam, such as a spread-out Bragg peak that provides a uniform beam having multiple energies. If various energy fields are to be used to treat the patient, it may not be accomplished using a broad beam. For example, a broad beam requires an ion beam compensator per treatment field customized per patient. This means there would be one compensator required for every angle, therefore, multiple compensators would have to be used to treat a patient. For instance, for at least every 4 degrees, a different compensator would have to be used. Treatment would have to be stopped and started using 90 different ion compensators to provide a 360 degree rotational proton radiation therapy. Another issue with using a broad beam is there is an undesired shape to the dose at the proximal edge of the targeted tumor.

### BRIEF DESCRIPTION OF THE DRAWINGS

FIG. 1 illustrates generally an example of a system, such as may include a particle therapy system controller, in accordance with an embodiment.

FIG. 2 illustrates generally an example of a radiation therapy system, such as may include a particle treatment system and an imaging acquisition device, in accordance with an embodiment.

FIG. 3 illustrates generally a particle treatment system that may include a radiation therapy output configured to provide a proton therapy beam, in accordance with an embodiment.

FIG. 4 illustrates generally radiation dose depths in human tissue for various types of particles, in accordance with an embodiment.

FIG. 5 illustrates generally a spread out Bragg Peak, in accordance with an embodiment.

FIG. 6 illustrates generally pencil beam scanning of an irregular shape volume from distal edge to proximal edge, in accordance with an embodiment.

FIG. 7 illustrates generally a diagram of an active scanning proton beam delivery system, in accordance with an embodiment.

FIGS. 8A-8C illustrate generally a beamlet delivery system as stationary spots, in accordance with an embodiment.

FIG. 9 illustrates generally a beamlet delivery system as line segments, in accordance with an embodiment.

FIG. 10A illustrates generally a linear spot delivery path on a grid, in accordance with an embodiment.

FIGS. 10B-10C illustrate generally spiral delivery paths on a grid, in accordance with an embodiment.

FIG. 11A illustrates a linear spot delivery path with differing spot sizes and a raster pattern, in accordance with an embodiment.

FIG. 11B illustrates a spiral spot delivery path with differing spot sizes, in accordance with an embodiment.

FIG. 12 illustrates a flowchart showing a technique for delivering a particle beam from a rotating gantry towards a target, in accordance with an embodiment.

FIG. 13 illustrates a flowchart showing a technique for generating a radiotherapy treatment plan to execute using a particle beam from a continuously rotating gantry towards a target, in accordance with an embodiment.

FIG. 14 illustrates an example schematic diagram of proton delivery to a target.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

Systems and techniques described herein may be used to generate a radiotherapy treatment plan to execute, for example using a particle beam from a continuously rotating gantry towards a target. An example technique may include identifying a target location within a tumor of a patient, providing a particle beam configured to deliver radiotherapy treatment to the tumor along a trajectory using at least two energies including a first energy and a second energy, the first energy greater than the second energy, and determining a first location along the trajectory past the target location and a second location before the target location along the trajectory. The technique may include determining a configuration for the particle beam to deliver the first energy to the first location and the second energy to the second location. In some examples, a radiotherapy treatment plan according to the configuration may be output.

The systems and techniques may be used to generate a plan to provide radiation therapy to a patient. The radiation therapy may be provided with a rotating gantry, for example by a particle beam affixed to the gantry. The gantry may continuously rotate while the particle beam applies a plurality of beamlets. The beamlets may be applied in a spiral pattern or a raster pattern on a target (e.g., a tumor or a portion of a tumor or other spot). In an example, rotating the gantry while delivering the particle beam may be inefficient (e.g., if dosage and penetration information for every degree or half degree is planned). In another example, rotating the gantry may introduce errors (e.g., if every few degrees are planned, such as every five or ten degrees). There are a number of advantages of providing rotational proton radiation therapy. First, instead of an undesired entrance dose at a small number of angles, the dose may be delivered from many angles.

The systems and techniques described herein may account for one or both of these issues by introducing a spiral or raster pattern for delivery of the beamlets. A spiral pattern may be used with planned angles at a range of degrees (e.g., five, ten, fifteen, etc.). In an example, a spiral pattern may include delivering the particle beam to a central portion of the target when at a highest error and to an outer portion of the target when at a lowest error. The amount of error may depend on angle difference between the actual gantry angle and the planned angle, for example with a higher error corresponding to a larger difference between angles, and a lower error corresponding to a smaller difference between angles.

In an example, a spiral pattern for applying a particle beam to a target may decrease time needed to complete a radiation therapy treatment. For example, beamlet size of beamlets delivered during the treatment may vary. To change size of the beamlets may cause disruption to treatment, for example by taking time or using energy. Using a raster type pattern may require multiple changes in beamlet size. Using the spiral pattern may allow for as few as a single change in beamlet size. For example, smaller beamlets may be used on an outside edge of a target, while larger beamlets may be used on an inside portion of the target.

Two locations may be used within a target for delivering energy from the particle beam (e.g., one that emits protons). The two locations may be located before or after a center of a target along a trajectory from the particle beam. A first location after the center may be used to deliver an energy greater than a second location before the center. The locations along the trajectory may be maintained as the gantry rotates, while the energy delivered may be changed.

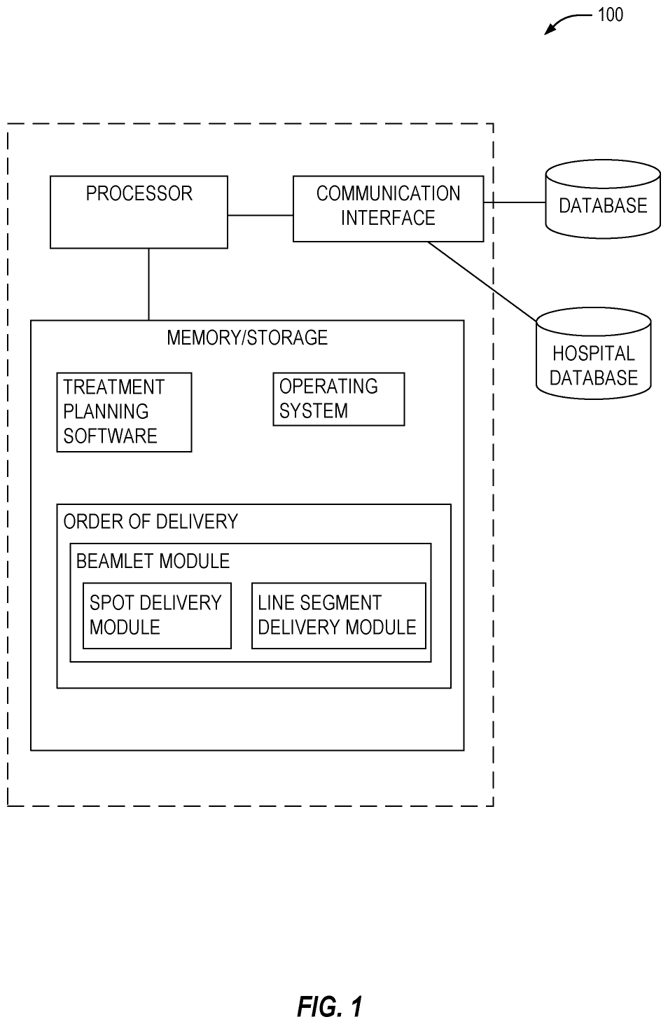

FIG. 1 illustrates generally an example of a system 100, such as may include a particle therapy system controller, in accordance with an embodiment. The system 100 may include a database or a hospital database. The particle therapy system controller may include a processor, communication interface, or memory. The memory may include treatment planning software, an operating system, or a delivery controller. The delivery controller may include a beamlet module for determining or planning spot delivery (e.g., using a spot delivery module) or line segment delivery (e.g., using a line segment delivery module).

In an example, the spot delivery module or the beamlet module may be configured to plan size of beamlets, location of a target or spot, or the like. The beamlet module may be used to determine an order of delivery of beamlets, for example in a spiral pattern as described herein. The order of delivery module may be in communication with the treatment planning software for planning delivery of beamlets. For example, the treatment planning software may be used to determine or plan gantry angle, gantry speed, beamlet size, spiral pattern (e.g., clockwise or counterclockwise), angle range for a particular spiral pattern (e.g., every ten degrees of the gantry rotation), or the like.

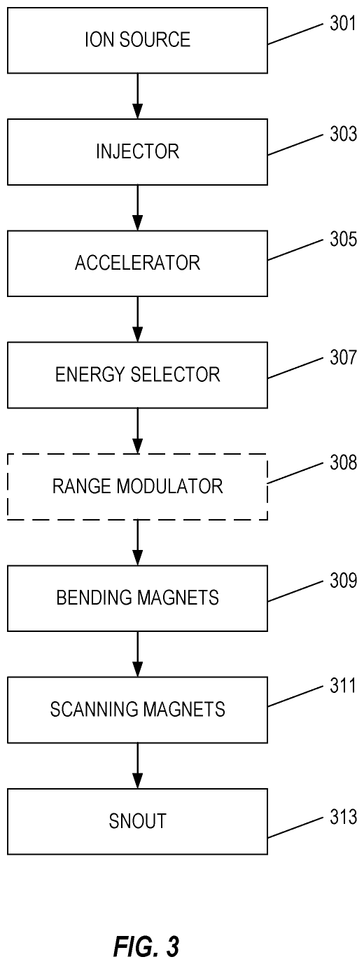

The processor may implement the plan, such as by communicating, via the communication interface or otherwise, to components used to implement the plan (e.g., to control devices or components, such as those described below with reference to FIG. 3). In an example, the communication interface may be used to retrieve stored information from a database or a hospital database (e.g., patient information, past procedure information for the patient or other patients, procedure instructions, information about particular devices or components, or the like).

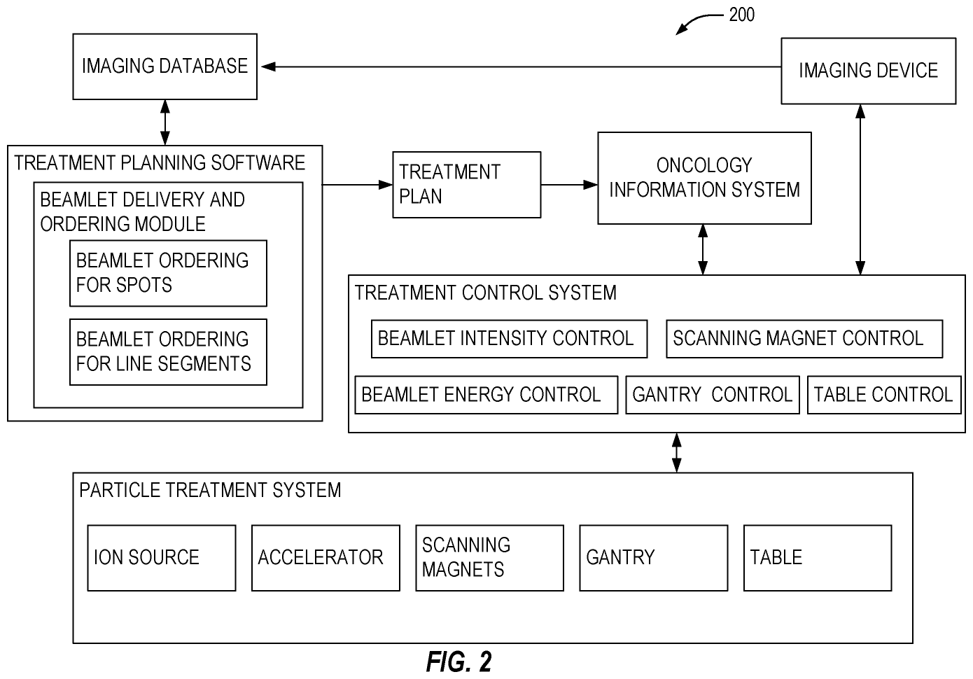

FIG. 2 illustrates generally an example of a radiation therapy system 200, such as may include a particle treatment system and an imaging acquisition device, in accordance with an embodiment. The particle treatment system includes an ion source, an accelerator, and scanning magnets, each of which is described in more detail below with respect to FIG. 3. The particle treatment system includes a gantry and a table, where the gantry may be mounted on the table, affixed to the table, or stabilized with respect to the table. The table may hold a patient. The gantry may be a rotating gantry, and may rotate with respect to the table (e.g., around the table) or with respect to the patient (and the table or a portion of the table may rotate with the gantry).

The particle treatment system may communicate with a treatment control system, which may be used to control actions of the particle treatment system. The treatment control system may communicate with an imaging acquisition device (e.g., to receive images taken by the imaging acquisition device or an imaging database) or an oncology information system. The oncology information system may provide treatment plan details to the treatment control system, such as received from treatment planning system. The treatment control system may use the treatment plan to control the particle treatment system (e.g., activate the gantry, the ion source, the accelerator, the scanning magnets, a particle beam, or the like). The treatment control system, for example, may include a beamlet intensity control, a beamlet energy control, a scanning magnet control, a table control, a gantry control, etc. In an example, the beamlet intensity control and the beamlet energy control may be used to activate a beamlet of a particular size or to target a particular location. The scanning magnetic control may be used to deliver beamlets according to the treatment plan, for example in a spiral pattern. The gantry control or the table control may be used to rotate the gantry.

The treatment planning software may include components such as a beamlet delivery and ordering module, with, for example, separate controls for beamlet ordering for spots or line segments. The treatment planning software is described in more detail above with respect to FIG. 1. The treatment planning software may access an imaging database to retrieve images or store information. When a treatment plan is completed, the treatment planning software may send the plan to an oncology information system for communication with the treatment control system.

FIG. 3 illustrates in an embodiment of a particle treatment system 300 that may include a radiation therapy output configured to provide a proton therapy beam. The particle treatment system 300 includes an ion source 301, an injector 303, an accelerator 305, an energy selector 307, a plurality of bending magnets 309, a plurality of scanning magnets 311, and a snout 313.

The ion source 301, such as a synchrotron (not shown) may be configured to provide a stream of particles, such as protons. The stream of particles is transported to an injector 303 that provides the charged particles with an initial acceleration using a Coulomb force. The particles are further accelerated by the accelerator 305 to about 10% of the speed of light. The acceleration provides energy to the particles, which determines the depth within tissue the particles may travel. The energy selector 307 (e.g., a range scatter) may be used to select the energies of the protons to be delivered to the patient. In an embodiment called passive scattering, an optional range modulator 308 (e.g., also called a ridge filter or a range modulation wheel) may be utilized to broaden the beam to fit the tumor. After selecting energies, a set of bending magnets 309 may be utilized to transport the stream of protons into a radiation therapy treatment room of a hospital. Further, scanning magnets 311 (e.g., x-y magnets) are used to spread the proton beam to, or trace, an exact image of the tumor shape. A snout 313 is used to further shape the proton beam. In various embodiments, the stream of particles may be composed of carbon ions, pions, or positively charged ions.

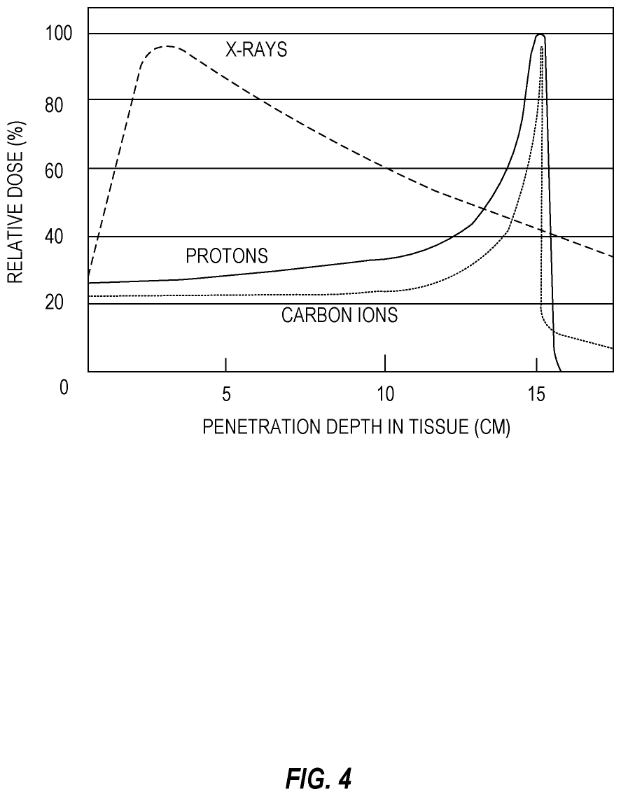

FIG. 4 provides an illustration of a comparison of radiation dose depths for various types of particles in human tissue. As shown, the relative depth of penetration into human tissue of photons (e.g., x-rays) versus protons versus carbon ions is provided (e.g., including any radiation dose provided at a distance beneath the surface, including secondary radiation or scatter). Each radiation dose is shown relative to the peak dose for a proton beam having a single energy which has been set to 100%.

The mono-energetic (e.g., single energy) proton beam indicates a plateau region starting at approximately 25% that gradually increases until approximately 10 cm depth in tissue where it rapidly increases to the Bragg Peak at 15 cm and then advantageously falls to zero within a short distance. No additional dose is delivered at the end of the Bragg peak.

The photon beam (e.g., labelled as X-rays) indicates the initial build up due to electron scatter (e.g., the primary means by which X-rays deliver dose to tissue is through transfer of energy to electrons in the tissue). This is followed by an exponential fall off, which continues past the distal edge of the target, which is at approximately 15 cm depth in the diagram. The x-ray beam has an entrance (skin) dose set to match that of the proton beam. With normalization (e.g., scaling) at 15 cm depth, the dose due to x-rays is at 40% of the dose provided by proton beam, while the x-ray beam has a peak dose of greater than 95% (“near” 100%) at approximately 3 cm depth. If the x-ray data is renormalized to achieve 100% dose at 15 cm, the peak dose at approximately 3 cm depth would be approximately 240%, in a location where dose is not desired (e.g., prior to the target). Therefore, with x-rays, a considerable amount of dose is delivered prior to the target and an appreciable amount of dose is delivered past the target.

The mono-energetic carbon beam shows a plateau region at the entrance dose that is lower than the proton beam. The carbon beam has a sharper Bragg Peak that falls more precipitously than the proton beam, but the carbon beam has a tail (e.g., known as a “spallation tail”, where some of the Carbon nuclei shatter in to Helium ions) that has approximately 10% additional dose, or less, past the desired target by several centimeters. The carbon ion beam has an undesired entrance and skin dose compared to the proton beam, but the carbon ion beam has a non-trivial dose delivered past the target.

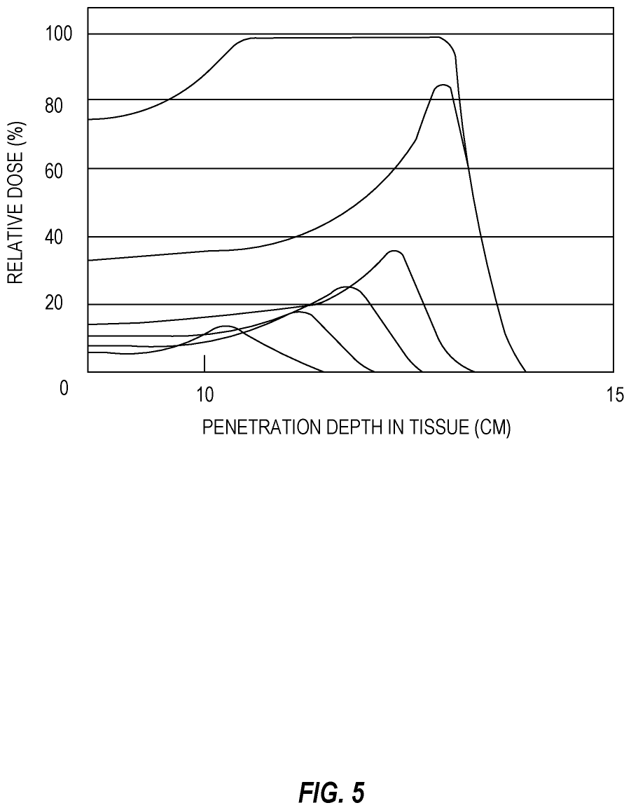

FIG. 5 provides an illustration of a spread-out Bragg peak (SOBP). The SOBP. displays a relative depth dose curve for the combination of a set of proton beams of various initial energies each of which has had some spread in energy (e.g., variable absorption of energy in tissue). The desired result of having a uniform dose for a target of a particular thickness. As shown, the target is shown with a proximal depth of approximately 10 cm, a distal depth of approximately 13 cm, and a target thickness of approximately 3 cm. Within the target, the dose is quite uniform (with an average normalized at 100%). The diagram does not start at 0 cm depth and is not explicitly showing the entrance (skin) dose, but the nature of the entrance region of proton beams is a relatively flat depth dose curve. Typically, the entrance (skin) dose will be approximately 70% of the target dose (e.g., shown at the far right edge of the x-axis). A SOBP may be obtained using a variety of approaches, including using a scattered proton beam with modulation of the energy (variable absorption) utilizing a variety of devices (e.g., a static ridge filter or a dynamic range modulation wheel), or by selection of a number of mono-energetic proton beams that do not undergo scatter.

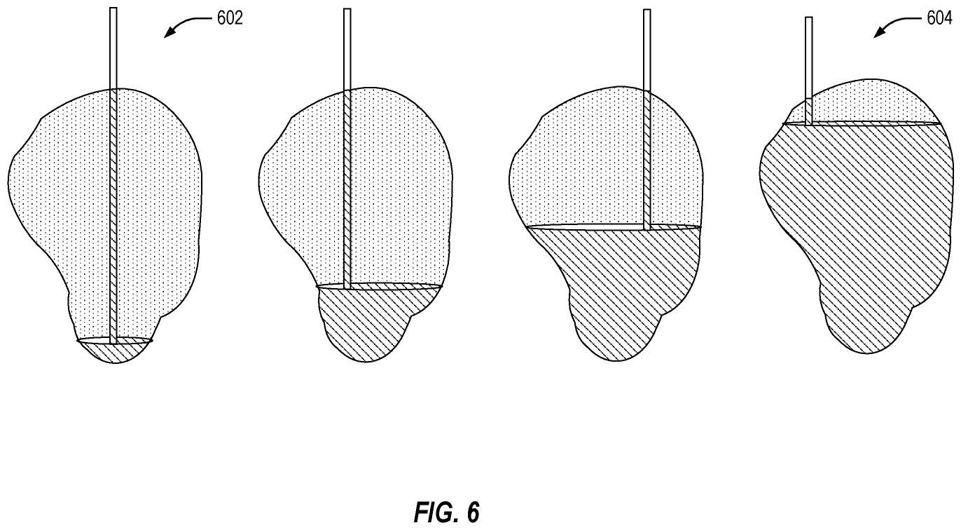

FIG. 6 provides an illustration of a Pencil Beam Scanning of an irregular shape volume from a distal edge (e.g., bottom) to a proximal (e.g., top) edge. As shown, the irregular shaped tumor volume is irradiated layers of protons. For example, a first time snapshot 602 shows a first layer of protons being delivered, and a later time snapshot 604 shows that most of the layers have been delivered. Each layer has its own cross-sectional area to which the protons having the same energy are delivered. The total radiation dose is provided as a layer-by-layer set of beamlets. Each layer of may have different energies. The most common means of specifying and delivering the set of beamlets to the cross-sectional area is to define and deliver beamlets having a constant diameter (“spot size”) to a selection of grid points on each layer. While the majority of the dose from the beamlet is delivered to the targeted layer, a significant amount of dose is delivered along the path to the targeted layer. The dose to proximal layers from beamlets defined for distal layers is accounted for in the specification of the beamlets defined for the proximal layers. The ability to individually specify the number of particles (e.g., the meterset) for a given beamlet ensures that each part of the volume being irradiate receives the desired dose.

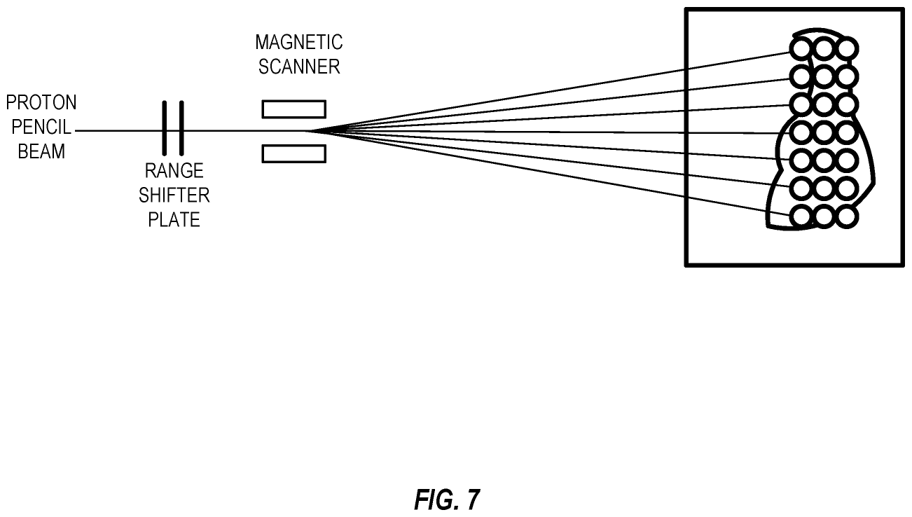

FIG. 7 provides an illustration of a diagrammatic representation of a typical active scanning proton beam delivery system. As shown, a single layer of a pencil beam scan is being delivered, with a grid of spots depicted on a patient in conjunction with a contour of the cross-sectional area to which particles are to be delivered. An incoming mono-energetic proton beamlet has a specified amount of its energy absorbed by the Range Shifter (e.g., in FIG. 7 it is a Range Shifter plate), resulting in a beamlet with the desired energy to achieve a certain depth for the Bragg Peak in the patient to treat the specified layer. A magnetic scanner, which has the ability to deflect the particles in both a vertical and a horizontal direction. The strength of the magnetic fields may be adjusted to control the deflection in the direction perpendicular to the magnetic field and the incoming beamlet. The rate at which the magnetic field strengths may be adjusted determines the rate at which the scanning may take place. For instance, the intensity of the proton beamlet in combination with the scanning rate determines how much dose may be delivered to a specific area (e.g., in FIG. 7, a “spot”) in a particular amount of time (e.g., particles/unit area). In theory, the magnetic field strengths may be adjusted independently of each other (in a fashion similar to the children's toy “Etch a Sketch®”, provided by Spin Master™, Toronto, Canada; with the pencil beamlet intensity being a variable not available in the children's toy). The most common scheme for scanning is to scan in one direction quickly and to scan in the perpendicular direction more slowly in a raster fashion, similar to how early televisions were controlled (e.g., Cathode Ray Tube (CRT), which use electrons instead of protons), but arbitrary patterns may be scanned (similar to the previously mentioned toy). Delivery of distinct spots is achieved by incrementing the scanning magnetic field strength and throttling the pencil beam intensity between increments.

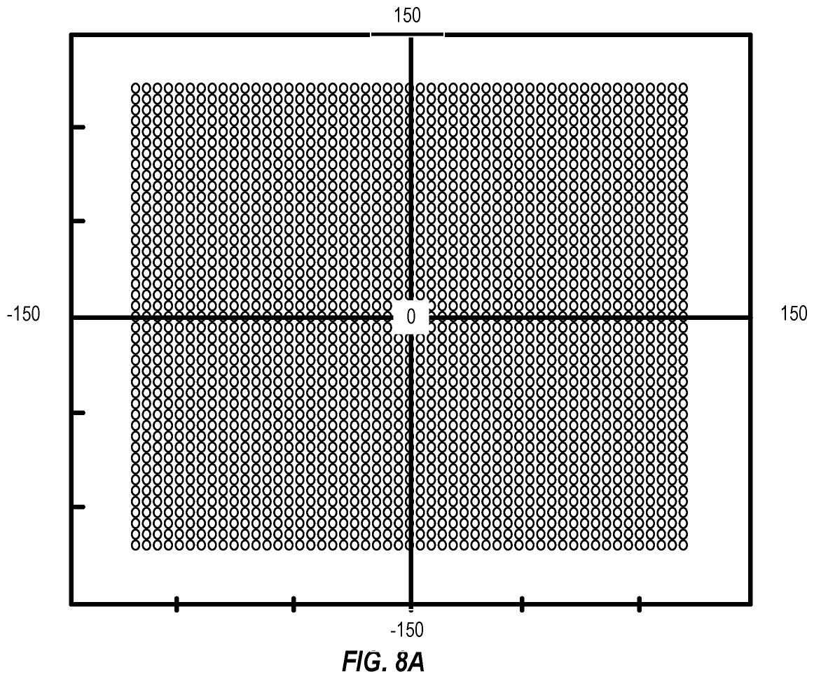

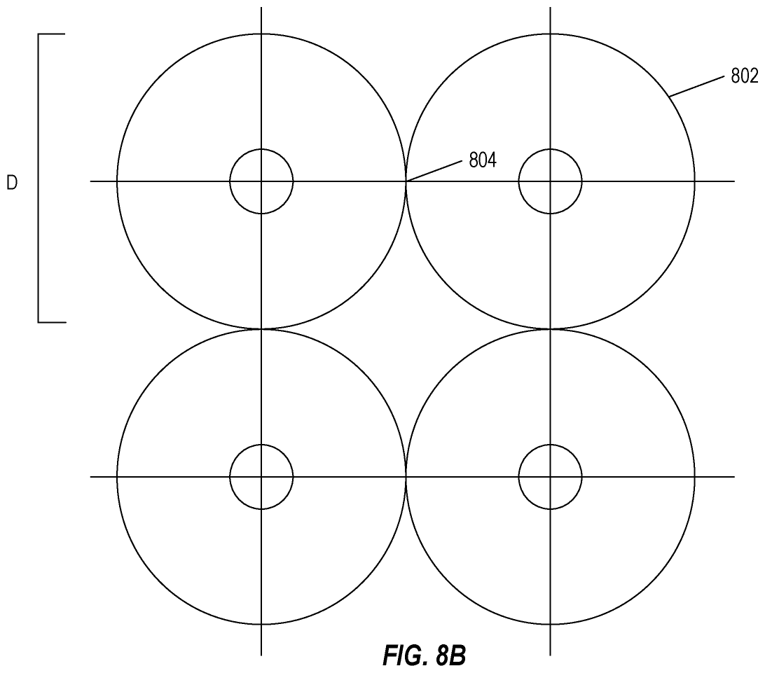

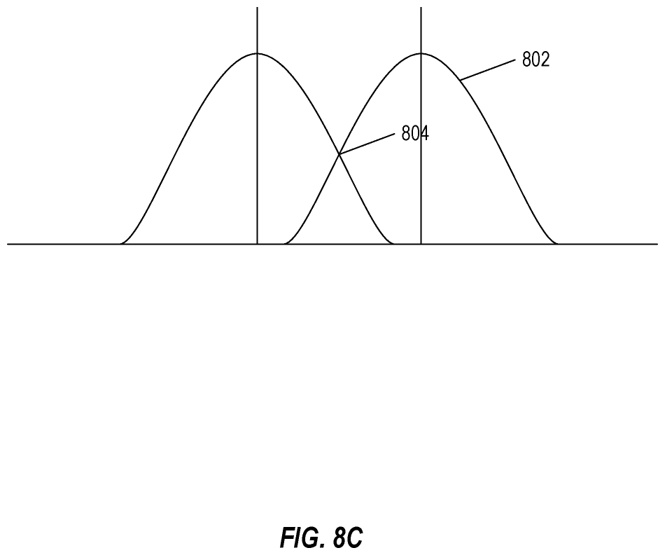

FIGS. 8A-8C illustrate generally a beamlet delivery system as stationary spots, in accordance with an embodiment. For illustrative purposes, when delivering beamlets to stationary spots, the spots are adjacent to one another on a Cartesian grid. The spots are nominally circular in shape having a size parameter (e.g., nominally a diameter) roughly equal to the grid spacing. In practice, spots are located next to one in a grid layout such that the edges of each spot abut against one another. The spot size diameter may be selected by the user based on machine capabilities. In an embodiment, small spots range from 2-4 millimeters, and large spots range from 8-12 millimeters in diameter. In an embodiment a small spot may be 3 millimeters, and a large spot may be 10 millimeters in diameter. As shown in FIG. 8A, the spots may be arranged as having an ellipsoid shape of approximately about 2 millimeters of the semi-minor axis and about 5 millimeters on the semi-major axis. For this particular example, the delivery of a set of beamlets may be provided at distinct spacing of 5 mm in both x-y directions.

FIG. 8B illustrates a grid having four adjacent spots, each spot (e.g., spot 802) having the same diameter D with radius ½D. The intensity pattern (e.g., particles delivered per unit volume) at a spot in practice may be approximated as a Gaussian function, as shown in FIG. 8C, which illustrates a Gaussian distribution of radiation dose, full width-half max. The size parameter (e.g., diameter) of the spot defines a “Full Width at Half Max” of the peak intensity at the center of two adjacent spots.

With a size parameter equal to the grid spacing, the beamlets delivered to adjacent spots overlap. A point 804, which is equidistant between spot A and spot B, for example, has a summed intensity (e.g., number of particles per unit area) with half the Gaussian distribution contributed by each spot (or, a volume when considering all three x-y-z dimensions). If the spots are of equal meterset (e.g., the same number of particles being delivered to every spot), the point 804 equidistant between spot A and spot B has the same intensity as the centers of the adjacent spots because of the summed intensity. This results in a highly uniform plane of dose delivery.

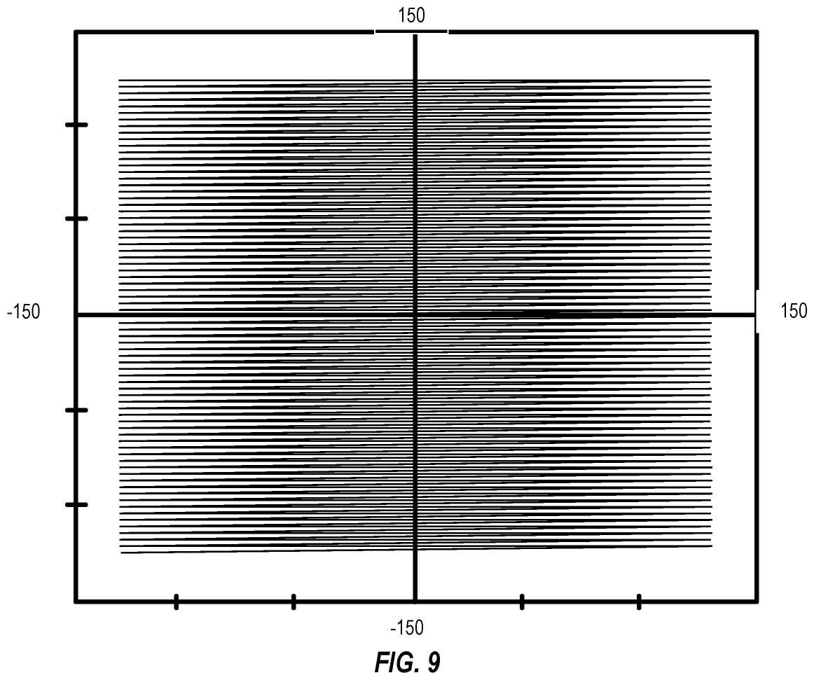

FIG. 9 provides an illustration of beamlet delivery as line segments. The delivery mode of line segments of particle beamlets sometimes referred to as line scanning is a type of scan mode that is linear. Each beamlet that is delivered to the target has a starting point and an ending point. As illustrated, a beamlet is continuously scanned from a spot on the right to a spot on the left. The size and spacing of the line segments allow for a uniform dose delivery. The scanning may be controlled in in any combination of continuous and/or incremented fashion, and independently control the particle beamlet intensity, generating an arbitrary pattern.

A proton broad beam, such as one created using scattering and utilizing a spread-out Bragg peak, provides a beam delivering relatively uniform dose to the entire target volume. If many fields are to be used to treat the patient, it may not be accomplished using proton broad beams. For example, a broad beam requires an ion block and an ion beam compensator per treatment field customized per patient. This means there would be one block and one compensator required for every angle, therefore, multiple blocks and compensators would have to be used to treat a patient. For example, for at least every 4 degrees, a different block and a different compensator would have to be used. A system would have to stop and start treatment using 90 different ion blocks and 90 different ion compensators to provide a 360 degree rotational proton radiation therapy. Even if a system used a multi-leaf collimator instead of ion blocks, the system would still need to use ion compensators. Another issue with using a broad beam is there is an undesired shape to the dose at the proximal edge of the targeted tumor due to the use of the ion compensator.

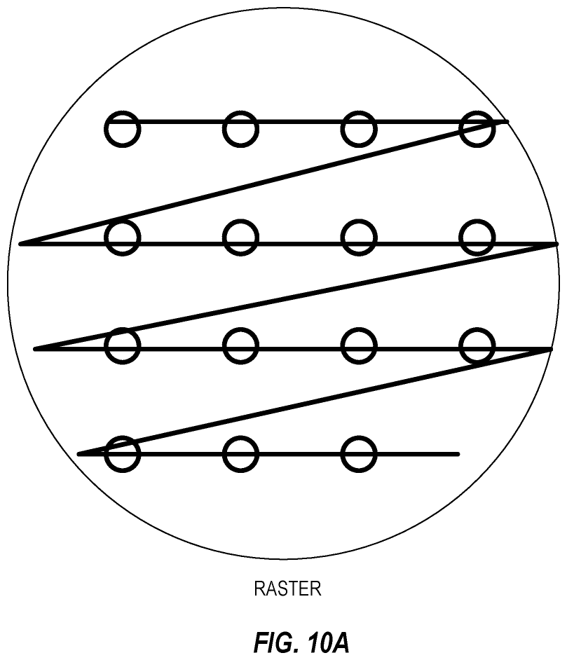

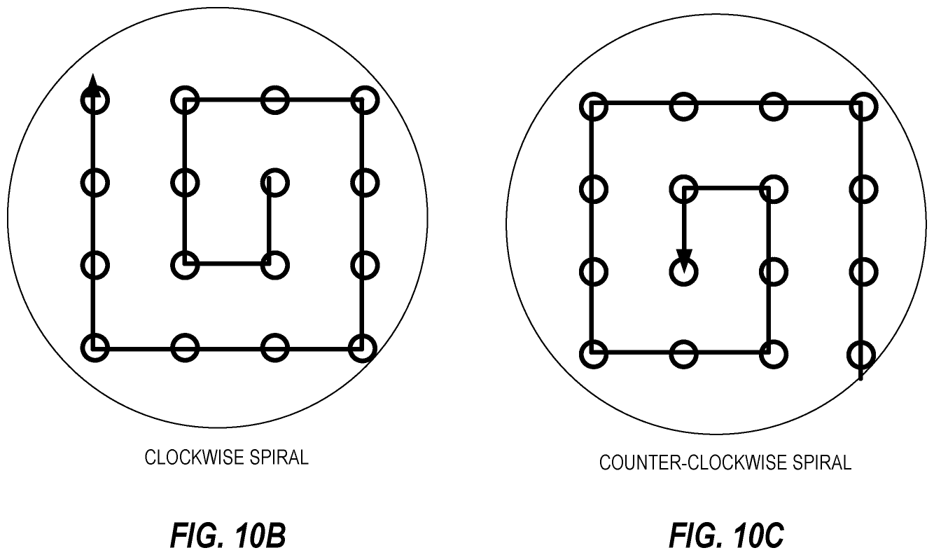

FIG. 10A illustrates generally a linear spot delivery path on a grid, in accordance with an embodiment. One form of particle therapy is spot-scanning. Spot-scanning allows for the delivery of complex dose distributions. With a rotating gantry, there are issues of spot placement of a particle beam because the spots are not delivered instantaneously, and as the gantry moves, the spots are delivered at an angle that is not exactly the planned nominal gantry angle. The system must deliver a plurality of beamlets, deliver using a certain particle energy, at a particular gantry angle, while the gantry rotates constantly at a nominally fixed rate (to avoid vibration and flexing). This must be accomplished to accurately reflect what was planned in the radiation therapy treatment plan, if the planning assumes a sampling of individual angles. The linear spot delivery path using a raster pattern shown in FIG. 10A illustrates one approach that may result in errors as the gantry rotates.

The raster pattern approach to treatment planning assumes that a plurality of beamlets will be delivered from a single angle constituting a static beam. More than one static beam may be defined as part of a treatment plan. To utilize existing treatment planning systems, proton arc therapy must be modelled as being delivered from a set of angles rather than a continuum of angles. While a system may attempt to model the delivery of each beamlet from a distinct angle during rotation, the added complexity, including machine specific behavior that might vary, from day to day makes this approach impractical. In practice, the delivery of the plurality of beamlets associated with a planned angle is delivered from the midpoint between the current planned angle and the previous planned angle through the current planned angle until the midpoint between the current planned angle and the next planned angle (potentially wrapping to the angle 360 degrees from the first/initial planned angle of the beginning of the arc).

FIGS. 10B-10C illustrate generally spiral delivery paths on a grid, in accordance with an embodiment. The spiral patterns shown in FIGS. 10B-10C minimize the errors resulting from a rotating gantry. The spiral patterns shown improve target accuracy and decrease radiation outside the target compared to the raster pattern shown in FIG. 10A while the gantry rotates.

The systems and methods described herein use proton arc therapy to optimize a radiation dose when delivering protons to certain spots. When delivering to certain spots, discrepancies may be minimized for what was planned versus what is actually delivered using the spiral pattern scan described herein. Unless the spots that are further from the isocentric axis are delivered while the gantry is closest to the current planned angle, the resulting actual spot location may be far from the intended spot location and the overall trajectory of the beamlet will differ significantly from the expected trajectory. Using a spiral scan minimizes the errors in the actual spot locations and minimizes the discrepancy between the expected and actual trajectories of the beamlets.

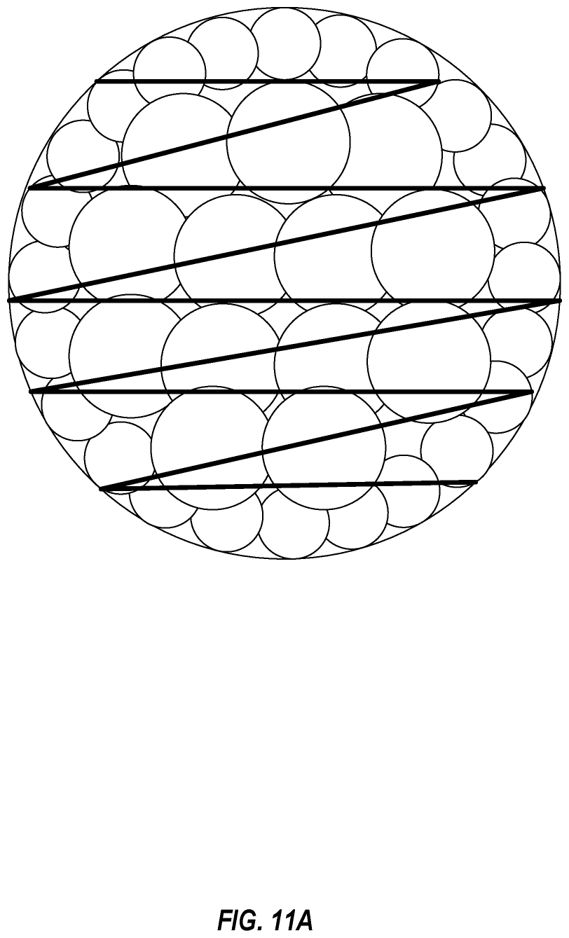

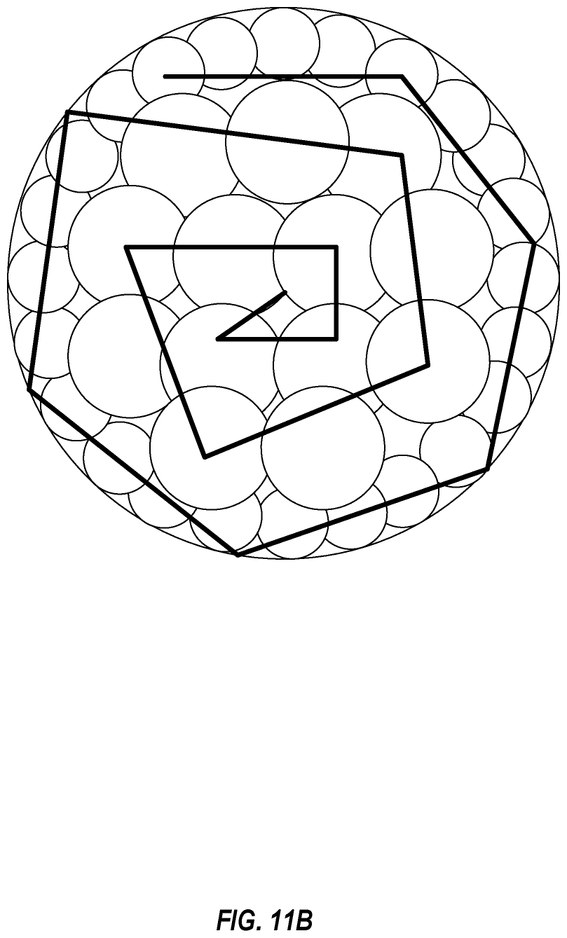

FIG. 11A illustrates a linear spot delivery path with differing spot sizes and a raster pattern, in accordance with an embodiment. FIG. 11B illustrates a spiral spot delivery path with differing spot sizes, in accordance with an embodiment.

The trade-off between a small and large spot is that to only deliver small beamlets to a small spot takes an inordinate amount of time to deliver radiation therapy. Therefore, to decrease the time, it is better to deliver the small beamlets to the outer edges/exterior of the tumor and deliver larger spots to the interior of the tumor. Changing spot size during delivery is a time consuming activity. When using the raster pattern of FIG. 11A for delivery of spots, any attempts to change the spot size when at the outer edges of the tumor result in a transition. As shown in FIG. 11A, each line on the grid involves two changes in spot size, resulting in dozens of transitions between spot sizes for the entire beam. By using the spiral delivery pattern shown in FIG. 11B, there may be as few as a single transition in spot sizes when going from the set of smaller spots treating the outer edges of the tumor to the set of larger spots treating the inner region of the tumor. Similarly, there may be as few as a single transition in spot sizes when going in the reverse, resulting in only two changes in spot size for the entire beam. In an example, the spiral pattern may be a two-dimensional spiral pattern, delivering dosage at each layer of the target.

In an example, beamlets may be delivered at the edges of an arc range may while the spiral is in the center of the target. For example, in an arc from 0 degrees to 10 degrees, the target may be planned as if the gantry was stationary at 5 degrees. In this example, the outside of the spiral occurs as the gantry approaches and leaves 5 degrees, while the center of the spiral occurs as the gantry leaves 0 degrees and as the gantry approaches 10 degrees. For example, starting at 0 degrees, the spiral may begin at the center of the target and spiral outward until ending (at an outward point of the spiral) around 5 degrees. Then, in an example, the spiral may reverse (e.g., move clockwise from 0 to 5 degrees, then counter-clockwise from 5 to 10 degrees, or vice versa) on the way back to the center of the target as the gantry moves from 5 to 10 degrees. The process may be repeated on a different layer of the target at another arc, for example from 10 to 20 degrees, etc., until the dose is completed.

When delivering a radiation treatment using particle arc therapy, specifying the delivery to be the equivalent of a number of fixed beams from a plurality of angles where each beam is to deliver a nominally uniform dose by using a plurality of energies creates the demand that near a given “nominal” beam angle many energies be delivered. This may be a challenge for a delivery system when the time to change energies is significant with respect to the time before the gantry has moved through a significant amount of arc angle. Further, it does not allow for using additional parameters available to the system that are easily adjusted during planning and optimization and that can be modulated very quickly at the time of delivery. Constraining the system to be the sum of a set of nominally uniform dose distributions may place higher linear energy transfer (LET) radiation near the edges of the tumor where the resulting higher effective, rather than just physical, dose may be nearer to an Organ at Risk and to Normal Tissue.

In an example, to mitigate these issues, the arc may be considered to be subdivided into a number of angles where for each angle, two further subdivisions are given, for example with one higher and one lower energy delivered to different locations within the tumor. For example, the higher energy peak may be delivered between the center of the tumor and a distal edge of the tumor (e.g., from a proton beam emitting the energy), while the lower energy peak may be placed between a proximal edge of the tumor and the center of the tumor. This arrangement may be used to place high LET radiation nearest the peak well within the tumor, even given uncertainty regarding the position of the tumor and the stopping power of the tissue through which the beams travel. This arrangement may be used to deliver substantial dose for each pair to the center of the tumor.

Individual beamlet weights in the beam may be initially chosen to achieve a nominally uniform dose across the width of the beam at the Bragg peak. A nominally uniform dose value may be set to a total desired dose to the tumor divided by the number of beams to be delivered throughout the arc. Because the dose of these beams is summed over the arc, there is a 1/x (x meaning radial distance from the isocenter in the direction of the arc) accumulation that causes the dose at the isocenter (the center of the tumor) to be higher than the dose further away from the isocenter (the edge of the tumor). To compensate for this, the individual beamlets at the center of the beam may be de-weighted (e.g., by a factor of r, where at the center the value of r is considered to be a specific non-zero value). Techniques may be used related to CT Filtered Back Projection (ramp filter), and utilized in algorithms for determining planned fluence in tomotherapy.

When constrained to an angle increment defined by the capabilities of the machine (for rate of gantry rotation and speed of energy switching), a set of deterministically and automatically defined beams may be generated. These beams may include individual dose distributions that may be calculated using one or more algorithms. Scaling factors for the individual beams may be calculated based on error minimization of the total arc calculated dose with respect to the desired dose. The error minimization may for example be inclusive of penalties identified in accordance with a clinicians' preferred trade-offs (e.g. dose uniformity, dose to organ at risk). This scaling factor optimization may include a linear optimization problem. The set of independent variables may be constrained to be in the hundreds for a system where the individual angle increment is on the order of approximately more than one and less than six degrees. This describes the scale of the optimization problem given realistic circumstances.

Where the above approach does not result in an acceptable dose distribution, the individual beamlet intensities may be varied from the above as a starting point. A linear optimization of those individual beamlet intensities may be performed with the individual dose distributions from each beamlet summed in total. The scale of the optimization problem in this example may be increased by a hundred-fold or a thousand-fold in terms of the number of independent variables.

The dose may be recalculated using the above determined values for a variety of values of positioning (uncertainty) and stopping power (uncertainty). A constraint may be used to ensure that results in the face of these uncertainties are acceptable (e.g., the plan generated is a robust plan). In the event that the results are not acceptable, separate optimizations for each of the discrete sets of positioning and stopping power used to establish error boundaries may be performed. For example, a round robin evaluation of the results for each of those solutions against the discrete sets of positioning and stopping power may be used. A linear optimization of the combination of solutions that achieves an optimal result in a robustness evaluation may be used to generate the final plan.

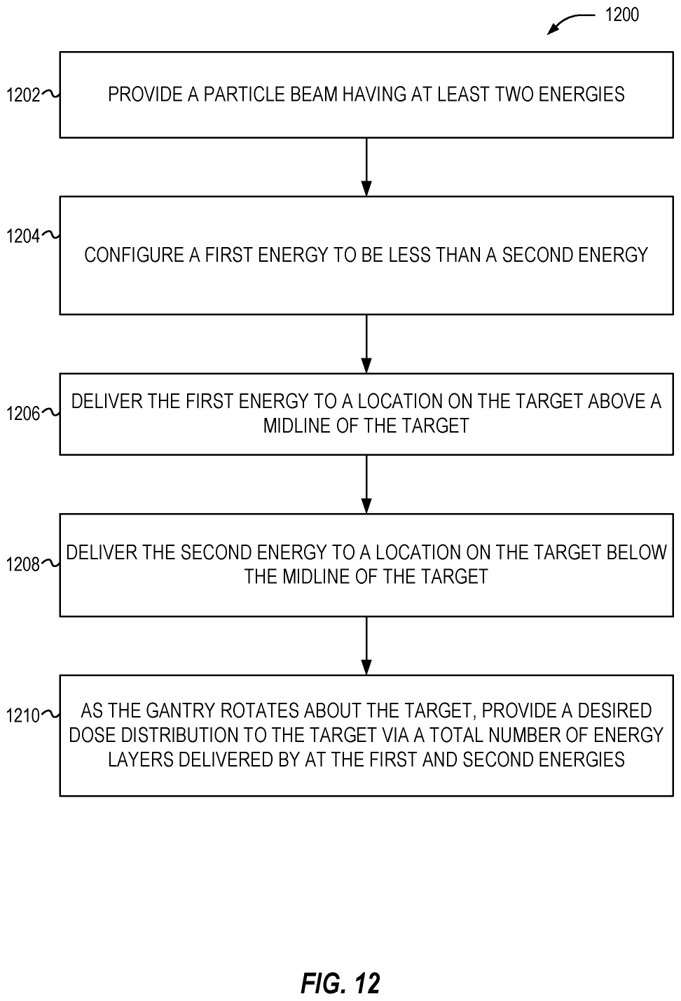

FIG. 12 illustrates a flowchart showing a technique 1200 for delivering a particle beam from a continuously rotating gantry towards a target, in accordance with an embodiment.

The technique 1200 includes an operation 1202 to provide a particle beam having at least two energies.

The technique 1200 includes an operation 1204 to configure a first energy to be less than a second energy.

The technique 1200 includes an operation 1206 to deliver the first energy to a location on the target above a midline of the target.

The technique 1200 includes an operation 1208 to deliver the second energy to a location on the target below the midline of the target.

The technique 1200 includes an operation 1210 to as the gantry rotates about the target, provide a desired dose distribution to the target via a total number of energy layers delivered by at the first and second energies.

The technique 1200 may include determining a robustness by a minimum error in dose distribution. In an example, the minimum error in the dose distribution is the actual dose distribution compared to the intended dose distribution. In an example, the dose distribution has an error input. In an example, the dose distribution error is least sensitive to the error input. In an example, the error input is associated with the stopping power values of the tissue.

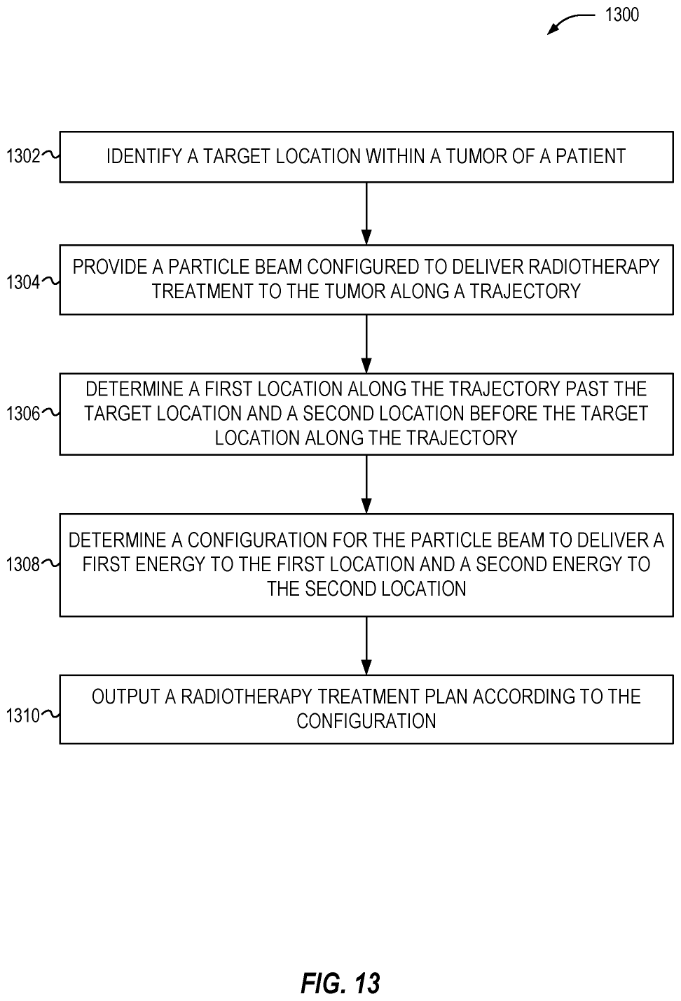

FIG. 13 illustrates a flowchart showing a technique 1300 for generating a radiotherapy treatment plan to execute using a particle beam from a continuously rotating gantry towards a target, in accordance with an embodiment.

The technique 1300 includes an operation 1302 to identify a target location within a tumor of a patient. The technique 1300 includes an operation 1304 to provide a particle beam configured to deliver radiotherapy treatment to the tumor along a trajectory. The particle beam may use at least two energies including a first energy and a second energy, the first energy greater than the second energy in some examples. The particle beam may be configured to deliver radiotherapy treatment to the tumor using a mini-ridge filter (MRF). In some examples, the particle beam emits protons.

The technique 1300 includes an operation 1306 to determine a first location along the trajectory past the target location and a second location before the target location along the trajectory. The technique 1300 includes an operation 1308 to determine a configuration for the particle beam to deliver an energy to the first location and an energy to the second location. For example, the first energy described above may be delivered at the first location and the second energy may be delivered at the second location (e.g., where the first energy is greater than the second energy).

The technique 1300 includes an operation 1310 to output a radiotherapy treatment plan according to the configuration. The radiotherapy treatment plan may include a planned rotation of the particle beam about the patient and a planned dose distribution to be delivered to the target via multiple sets of two energies delivered by the particle beam at a particular rotation angles. For example, the radiotherapy treatment plan may include a plan to deliver the first energy at the first location at a first offset of a particular rotation angle and a plan to deliver the second energy at the second location at a second offset of the particular rotation angle. In some examples, the first offset is between a quarter of a degree and five degrees before the particular rotation angle, and the second offset is between a quarter of a degree and five degrees after the particular rotation angle. Operation 1310 may include displaying the radiotherapy treatment plan on a user interface. The radiotherapy treatment plan may include delivering treatment according to a raster or spiral pattern. In some examples, the radiotherapy treatment plan requires a robustness factor, the robustness factor including a minimum error in dose distribution defined by comparing an actual dose distribution to an intended dose distribution.

The technique 1300 may include generating the radiotherapy treatment plan using inverse planning based on constraints including the first energy, the second energy, the first location, the second location, and a total dosage to deliver to the target. Generating the radiotherapy treatment plan may be performed automatically (e.g., using a processor) based on the constraints (e.g., in a fully constrained example). This automatic generation may replace a manual process where a clinician must construct a plan (e.g., in an under-constrained example). The technique 1300 may include configuring the particle beam to deliver the first energy to the first location and the second energy to the second location according to the radiotherapy treatment plan.

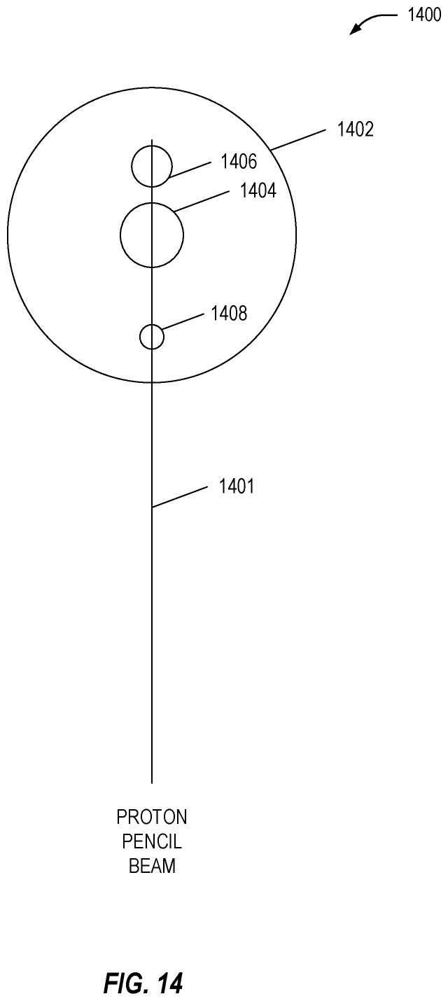

FIG. 14 illustrates an example schematic diagram 1400 of proton delivery to a target 1402. The diagram 1400 shows the target 1402, including a center 1404 of the target. A proton beam may deliver protons along a trajectory 1401 to the target 1402. The protons may be delivered at a first location 1406 and at a second location 1408, along the trajectory 1401. In some examples, protons are delivered to the first location 1406 at a greater energy than at the second location 1408.

In some examples, the proton beam may rotate about the target 1402. The diagram 1400 may illustrate a snapshot along this rotation. While the first location 1406 and the second location 1408 are shown along the trajectory 1401 at this snapshot, in reality they may be delivered at different angles of rotation, and thus be offset from each other or from a center angle (e.g., by a quarter of a degree, by five degrees, by ten degrees, etc.).

Each of the non-limiting examples described in this document may stand on its own, or may be combined in various permutations or combinations with one or more of the other examples.

Example 1 is a method of delivering a particle beam from a continuously rotating gantry towards a target, the method comprising: providing a particle beam having at least two energies; configuring a first energy to be less than a second energy; delivering the first energy to a location on the target above a midline of the target; delivering the second energy to a location on the target below the midline of the target; and as the gantry rotates about the target, providing a desired dose distribution to the target via a total number of energy layers delivered by at the first and second energies.

In Example 2, the subject matter of Example 1 includes, wherein robustness is determined by the minimum error in dose distribution.

In Example 3, the subject matter of Example 2 includes, wherein the minimum error in the dose distribution is the actual dose distribution compared to the intended dose distribution.

In Example 4, the subject matter of Example 3 includes, wherein the dose distribution has an error input.

In Example 5, the subject matter of Example 4 includes, wherein the dose distribution error is least sensitive to the error input.

In Example 6, the subject matter of Example 5 includes, wherein the error input is associated with the stopping power values of the tissue.

In Example 7, the subject matter of Examples 1-6 includes, wherein the desired dose distribution is robust in the face of uncertainties.

Example 8 is a method for generating a radiotherapy treatment plan to execute using a particle beam from a continuously rotating gantry towards a target, the method comprising: identifying a target location within a tumor of a patient; providing a particle beam configured to deliver radiotherapy treatment to the tumor along a trajectory using at least two energies including a first energy and a second energy, the first energy greater than the second energy; determining a first location along the trajectory past the target location and a second location before the target location along the trajectory; determining a configuration for the particle beam to deliver the first energy to the first location and the second energy to the second location; and outputting a radiotherapy treatment plan according to the configuration.

In Example 9, the subject matter of Example 8 includes, wherein the radiotherapy treatment plan includes a planned rotation of the particle beam about the patient and a planned dose distribution to be delivered to the target via multiple sets of two energies delivered by the particle beam at a particular rotation angles.

In Example 10, the subject matter of Example 9 includes, wherein the radiotherapy treatment plan includes a plan to deliver the first energy at the first location at a first offset of a particular rotation angle and a plan to deliver the second energy at the second location at a second offset of the particular rotation angle.

In Example 11, the subject matter of Example 10 includes, wherein the first offset is between a quarter of a degree and five degrees before the particular rotation angle, and wherein the second offset is between a quarter of a degree and five degrees after the particular rotation angle.

In Example 12, the subject matter of Examples 8-11 includes, configuring the particle beam to deliver the first energy to the first location and the second energy to the second location according to the radiotherapy treatment plan.

In Example 13, the subject matter of Examples 8-12 includes, wherein outputting the radiotherapy treatment plan includes displaying the radiotherapy treatment plan on a user interface.

In Example 14, the subject matter of Examples 8-13 includes, wherein the radiotherapy treatment plan includes delivering treatment according to a raster or spiral pattern.

In Example 15, the subject matter of Examples 8-14 includes, wherein the particle beam is configured to deliver radiotherapy treatment to the tumor using a mini-ridge filter (MRF).

In Example 16, the subject matter of Examples 8-15 includes, wherein the particle beam emits protons.

In Example 17, the subject matter of Examples 8-16 includes, wherein the radiotherapy treatment plan requires a robustness factor, the robustness factor including a minimum error in dose distribution defined by comparing an actual dose distribution to an intended dose distribution.

Example 18 is at least one machine-readable medium including instructions for generating a radiotherapy treatment plan to execute using a particle beam from a continuously rotating gantry towards a target, which when executed cause processing circuitry to perform operations to: identify a target location within a tumor of a patient; provide a particle beam configured to deliver radiotherapy treatment to the tumor along a trajectory using at least two energies including a first energy and a second energy, the first energy greater than the second energy; determine a first location along the trajectory past the target location and a second location before the target location along the trajectory; determine a configuration for the particle beam to deliver the first energy to the first location and the second energy to the second location; and output a radiotherapy treatment plan according to the configuration.

In Example 19, the subject matter of Example 18 includes, wherein the radiotherapy treatment plan includes a planned rotation of the particle beam about the patient and a planned dose distribution to be delivered to the target via multiple sets of two energies delivered by the particle beam at a particular rotation angles.

In Example 20, the subject matter of Example 19 includes, wherein the radiotherapy treatment plan includes a plan to deliver the first energy at the first location at a first offset of a particular rotation angle and a plan to deliver the second energy at the second location at a second offset of the particular rotation angle.

In Example 21, the subject matter of Example 20 includes, wherein the first offset is between a quarter of a degree and five degrees before the particular rotation angle, and wherein the second offset is between a quarter of a degree and five degrees after the particular rotation angle.

In Example 22, the subject matter of Examples 18-21 includes, wherein the instructions, when executed, further cause the processing circuitry to configure the particle beam to deliver the first energy to the first location and the second energy to the second location according to the radiotherapy treatment plan.

In Example 23, the subject matter of Examples 18-22 includes, wherein to output the radiotherapy treatment plan, the instructions, when executed, further cause the processing circuitry to display the radiotherapy treatment plan on a user interface.

In Example 24, the subject matter of Examples 18-23 includes, wherein the radiotherapy treatment plan includes delivering treatment according to a raster or spiral pattern.

In Example 25, the subject matter of Examples 18-24 includes, wherein the particle beam is configured to deliver radiotherapy treatment to the tumor using a mini-ridge filter (MRF).

In Example 26, the subject matter of Examples 18-25 includes, wherein the particle beam emits protons.

In Example 27, the subject matter of Examples 18-26 includes, wherein the radiotherapy treatment plan requires a robustness factor, the robustness factor including a minimum error in dose distribution defined by comparing an actual dose distribution to an intended dose distribution.

Example 28 is at least one machine-readable medium including instructions that, when executed by processing circuitry, cause the processing circuitry to perform operations to implement of any of Examples 1-27.

Example 29 is an apparatus comprising means to implement of any of Examples 1-27.

Example 30 is a system to implement of any of Examples 1-27.

Example 31 is a method to implement of any of Examples 1-27.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1. A method for generating a radiotherapy treatment plan to execute using a particle beam from a continuously rotating gantry towards a target, the method comprising:
identifying a target location within a tumor of a patient;
providing a particle beam configured to deliver radiotherapy treatment to the tumor along a trajectory using at least two energies including a first energy and a second energy, the first energy greater than the second energy;
determining a first location along the trajectory past the target location and a second location before the target location along the trajectory;
determining a configuration for the particle beam to deliver the first energy to the first location and the second energy to the second location; and
outputting a radiotherapy treatment plan according to the configuration.

2. The method of claim 1, wherein the radiotherapy treatment plan includes a planned rotation of the particle beam about the patient and a planned dose distribution to be delivered to the target via multiple sets of two energies delivered by the particle beam at a particular rotation angles.

3. The method of claim 2, wherein the radiotherapy treatment plan includes a plan to deliver the first energy at the first location at a first offset of a particular rotation angle and a plan to deliver the second energy at the second location at a second offset of the particular rotation angle.

4. The method of claim 3, wherein the first offset is between a quarter of a degree and five degrees before the particular rotation angle, and wherein the second offset is between a quarter of a degree and five degrees after the particular rotation angle.

5. The method of claim 1, further comprising configuring the particle beam to deliver the first energy to the first location and the second energy to the second location according to the radiotherapy treatment plan.

6. The method of claim 1, wherein outputting the radiotherapy treatment plan includes displaying the radiotherapy treatment plan on a user interface.

7. The method of claim 1, wherein the radiotherapy treatment plan includes delivering treatment according to a raster or spiral pattern.

8. The method of claim 1, wherein the particle beam is configured to deliver radiotherapy treatment to the tumor using a mini-ridge filter (MRF).

9. The method of claim 1, wherein the particle beam emits protons.

10. The method of claim 1, wherein the radiotherapy treatment plan requires a robustness factor, the robustness factor including a minimum error in dose distribution defined by comparing an actual dose distribution to an intended dose distribution.

11. At least one machine-readable medium including instructions for generating a radiotherapy treatment plan to execute using a particle beam from a continuously rotating gantry towards a target, which when executed cause processing circuitry to perform operations to:
identify a target location within a tumor of a patient;
provide a particle beam configured to deliver radiotherapy treatment to the tumor along a trajectory using at least two energies including a first energy and a second energy, the first energy greater than the second energy;
determine a first location along the trajectory past the target location and a second location before the target location along the trajectory;
determine a configuration for the particle beam to deliver the first energy to the first location and the second energy to the second location; and
output a radiotherapy treatment plan according to the configuration.

12. The at least one machine-readable medium of claim 11, wherein the radiotherapy treatment plan includes a planned rotation of the particle beam about the patient and a planned dose distribution to be delivered to the target via multiple sets of two energies delivered by the particle beam at a particular rotation angles.

13. The at least one machine-readable medium of claim 12, wherein the radiotherapy treatment plan includes a plan to deliver the first energy at the first location at a first offset of a particular rotation angle and a plan to deliver the second energy at the second location at a second offset of the particular rotation angle.

14. The at least one machine-readable medium of claim 13, wherein the first offset is between a quarter of a degree and five degrees before the particular rotation angle, and wherein the second offset is between a quarter of a degree and five degrees after the particular rotation angle.

15. The at least one machine-readable medium of claim 11, wherein the instructions, when executed, further cause the processing circuitry to configure the particle beam to deliver the first energy to the first location and the second energy to the second location according to the radiotherapy treatment plan.

16. The at least one machine-readable medium of claim 11, wherein to output the radiotherapy treatment plan, the instructions, when executed, further cause the processing circuitry to display the radiotherapy treatment plan on a user interface.

17. The at least one machine-readable medium of claim 11, wherein the radiotherapy treatment plan includes delivering treatment according to a raster or spiral pattern.

18. The at least one machine-readable medium of claim 11, wherein the particle beam is configured to deliver radiotherapy treatment to the tumor using a mini-ridge filter (MRF).

19. The at least one machine-readable medium of claim 11, wherein the particle beam emits protons.

20. The at least one machine-readable medium of claim 11, wherein the radiotherapy treatment plan requires a robustness factor, the robustness factor including a minimum error in dose distribution defined by comparing an actual dose distribution to an intended dose distribution.
